(19) United States
(12) Patent Application Publication
(10) Pub. No.: US 2025/0180512 A1
(43) Pub. Date: Jun. 5, 2025

(54) HIGH-THROUGHPUT MULTIPLEXING ELECTROPHORETIC GEL APPARATUS AND RELATED METHODS

(71) Applicants: Cameron O. Zadeh, Greenville, SC (US); Marc R. Birtwistle, Central, SC (US); Jonah R. Huggins, Central, SC (US)

(72) Inventors: Cameron O. Zadeh, Greenville, SC (US); Marc R. Birtwistle, Central, SC (US); Jonah R. Huggins, Central, SC (US)

(21) Appl. No.: 19/045,930

(22) Filed: Feb. 5, 2025

Related U.S. Application Data
(62) Division of application No. 17/970,669, filed on Oct. 21, 2022.
(60) Provisional application No. 63/270,436, filed on Oct. 21, 2021.

(51) Int. Cl. G01N 27/447; G01N 33/68

(52) U.S. Cl. CPC G01N 27/44756; G01N 27/44721; G01N 27/44743; G01N 27/44747; G01N 33/6803

(57) ABSTRACT

A high-throughput multiplexing electrophoretic gel system is disclosed. The system includes a gel casting device which includes an interior, gel casting chamber by which a polymerized gel layer is formed. The polymerized gel layer includes a plurality of integrally formed sample loading wells. The wells are aligned to be simultaneously loaded with samples via an automated microliter multi-pipette sample loader. The sample-loaded gel layer is adapted to undergo immersed horizontal electrophoresis separation.

### I. RELATED APPLICATIONS

The present application is a divisional of U.S. Non-provisional patent application Ser. No. 17/970,669, filed on Oct. 21, 2022, which claims priority to U.S. Provisional Patent Application No. 63/270,436, filed on Oct. 21, 2021, the disclosures all of which are hereby incorporated by reference in their entireties.

### II. FIELD OF THE INVENTION

This application discloses claims and embodiments generally related to gel electrophoresis for analyzing biomolecular sample materials, and more particularly, to an improved apparatus, system, and methods for the simultaneous loading of a plurality of samples for electrophoretic separation of molecules.

### III. BACKGROUND OF THE INVENTION

Western Blotting is a technique and process used in cell and molecular biology to identify proteins from a mixture of proteins extracted from cells. Conventional laboratory equipment utilized to perform western blotting suffers from a number of drawbacks. For example, many known systems require long run times, cannot facilitate high lysate numbers, and/or require very large sample volumes to produce larger sample sizes. Most know systems that produce western blotting higher throughput are complex and consequently expensive, thus rendering them unaffordable for man smaller laboratories, colleges, and research organizations.

Currently there exists in the art, and this industry, a versatile apparatus that is compatible for use with standard western blotting systems which requires only a relatively small sample volume to produce results for a large number of samples.

A search of the prior art did not disclose any patents or publications that read directly on the claims of the instant invention; however, following references were considered related:

U.S. Pat. No. 11,112,415 B2, issued in the name of Fournier et al.;

U.S. Pat. No. 6,562,213 B1, issued in the name of Cabilly et al.;

U.S. Pat. No. 7,122,104 B2, issued in the name of Cabilly et al.;

U.S. Patent Publication No. 2021/0246440 A1, published in the name of St. Onge et al.;

Zadeh, C. O. et al. Mesowestern Blot: Simultaneous Analysis of Hundreds of Sub-Microliter Lysates. http://biorxiv.org/lookup/doi/10.1101/2021.11.07.467614 (2021) doi: 10.1101/2021.11.07.467614; https://pubs.acs.org/doi/full/10.1021/acsomega. 2c02201;

Moritz, C. P. 40 years Western blotting: A scientific birthday toast. J. Proteomics 212, 103575 (2020);

Hughes, A. J. et al. Single-cell western blotting. Nat. Methods 11, 749-55 (2014);

Nguyen, U., Squaglia, N., Boge, A. & Fung, P. A. The Simple Western™: a gel-free, blot-free, hands-free Western blotting reinvention. Nat. Methods 8, v-vi (2011);

Ciaccio, M. F., Wagner, J. P., Chuu, C.-P. Lauffenburger, D. A. & Jones, R. B. Systems analysis of EGF receptor signaling dynamics with microwestern arrays. Nat. Methods 7, 148-55 (2010);

Ciaccio, M. F. & Jones, R. B. Microwestern Arrays for Systems-Level Analysis of SH2 Domain-Containing Proteins. Methods Mol Biol 453-473 (2017) doi: 10.1007/978-1-4939-6762-9_27;

Towbin, H., Staehelin, T. & Gordon, J. Electrophoretic transfer of proteins from polyacrylamide gels to nitrocellulose sheets: procedure and some applications. Proc. Natl. Acad. Sci. 76, 4350-4354 (1979);

Uhlen, M. et al. A proposal for validation of antibodies. Nat. Methods 13, 823-7 (2016);

Björling, E. & Uhlen, M. Antibodypedia, a portal for sharing antibody and antigen validation data. Mol. Cell. Proteomics MCP 7, 2028-37 (2008);

Bourbeillon, J. et al. Minimum information about a protein affinity reagent (MIAPAR). Nat. Biotechnol. 28, 650-3 (2010);

Tibes, R. et al. Reverse phase protein array: validation of a novel proteomic technology and utility for analysis of primary leukemia specimens and hematopoietic stem cells. Mol. Cancer Ther. 5, 2512-21 (2006);

Hennessy, B. T. et al. A Technical Assessment of the Utility of Reverse Phase Protein Arrays for the Study of the Functional Proteome in Non-microdissected Human Breast Cancers. Clin. Proteomics 6, 129-51 (2010);

Earley, M. C. et al. Report from a workshop on multianalyte microsphere assays. Cytometry 50, 239-42 (2002);

He, J. Practical Guide to ELISA Development. in The Immunoassay Handbook 381-393 (Elsevier, 2013). doi: 10.1016/B978-0-08-097037-0.00025-7;

Engvall, E. & Perlmann, P. Enzyme-linked immunosorbent assay, Elisa. 3. Quantitation of specific antibodies by enzyme-labeled anti-immunoglobulin in antigen-coated tubes. J. Immunol. Baltim. Md 1950 109, 129-135 (1972);

Aebersold, R. & Mann, M. Mass-spectrometric exploration of proteome structure and function. Nature 537, 347-55 (2016);

Wilhelm, M. et al. Mass-spectrometry-based draft of the human proteome. Nature 509, 582-7 (2014);

Shevchenko, A., Tomas, H., Havlis, J., Olsen, J. V. & Mann, M. In-gel digestion for mass spectrometric characterization of proteins and proteomes. Nat. Protoc. 1, 2856-60 (2006);

Bittremieux, W. et al. Quality control in mass Quality spectrometry-based proteomics. Mass Spectrom. Rev. 37, 697-711 (2018);

Yates, J. R., Ruse, C. I. & Nakorchevsky, A. Proteomics by Mass Spectrometry: Approaches, Advances, and Applications. Annu. Rev. Biomed. Eng. 11, 49-79 (2009);

Timp, W. & Timp, G. Beyond mass spectrometry, the next step in proteomics. Sci. Adv. 6, eaax8978 (2020);

Handler, D. C. et al. The Art of Validating Quantitative Proteomics Data. PROTEOMICS 18, 1800222 (2018);

Kang, C.-C. et al. Single cell-resolution western blotting. Nat. Protoc. 11, 1508-30 (2016);

Treindl, F. et al. A bead-based western for high-throughput cellular signal transduction analyses. Nat. Commun. 7, 12852 (2016);

Koch, R. J. et al. Validating Antibodies for Quantitative Western Blot Measurements with Microwestern Array. Sci. Rep. 8, 11329 (2018);

Bouhaddou, M. et al. A mechanistic pan-cancer pathway model informed by multi-omics data interprets stochastic cell fate responses to drugs and mitogens. PLOS Comput. Biol. 14, (2018);

Han, Y., Thomas, C. T., Wennersten, S. A., Lau, E. & Lam, M. P. Y. Shotgun Proteomics Sample Processing Automated by an Open-Source Lab Robot. J. Vis. Exp. 63092 (2021) doi: 10.3791/63092;

Councill, E. E. A. W. et al. Adapting a Low-Cost and Open-Source Commercial Pipetting Robot for Nanoliter Liquid Handling. SLAS Technol. Transl. Life Sci. Innov. 26, 311-319 (2021);

Villanueva-Cañas, J. L. et al. Implementation of an open-source robotic platform for SARS-COV-2 testing by real-time RT-PCR. PLOS ONE 16, e0252509 (2021);

Storch, M., Haines, M. C. & Baldwin, G. S. DNA-BOT: a low-cost, automated DNA assembly platform for synthetic biology. Synth. Biol. 5, ysaa010 (2020);

Liu, X., Gygi, S. P. & Paulo, J. A. A Semiautomated Paramagnetic Bead-Based Platform for Isobaric Tag Sample Preparation. J. Am. Soc. Mass Spectrom. 32, 1519-1529 (2021);

Janes, K. A. An analysis of critical factors for quantitative immunoblotting. Sci. Signal. 8, rs2 (2015);

Western Blotting Market | 2021-26 | Industry Share, Size, Growth-Mordor Intelligence. https://www.mordorintelligence. com/industry-reports/western-blotting-market;

Itd, M. D. F. Western Blotting Market Size, Share, Trends Growth | 2021 to 2026. Market Data Forecast http://www.marketdataforecast.com/;

Western Blotting Market—Global Forecast to 2026 | MarketsandMarkets; https://www.marketsandmarkets.com/Market-Reports/western-blotting-market-235810711. html; and

Western Blotting Market Size & Share | Global Industry Report, 2025. https://www.grandviewresearch.com/industry-analysis/western-blotting-market.

This application presents claims and embodiments that fulfill a need or needs not yet satisfied by the products, inventions and methods previously or presently available. In particular, the claims and embodiments disclosed herein describe a high-throughput multiplexing system for conducting electrophoresis separation of molecules, the system comprising a gel casting device and a horizontal electrophoresis tank, the casting device comprises a top portion and a base, the top portion detachably connected to the base and forming an interior, gel casting chamber; the gel casting device further comprises at least one loading port through which a gel solution is introduced into the casting chamber, and wherein the base includes a plurality of protrusions interpolating the gel solution as the gel solution is loaded into the casting chamber, thereby forming a plurality of sample loading wells after polymerization of the gel, the wells each being simultaneously auto-loaded with approximately 1.00 μl volume of a liquid sample, the horizontal electrophoresis tank is filled with a buffer solution to a level so as to immerse the sample-loaded polymerized gel, and the tank is configured to enable the sample-loaded polymerized gel to undergo horizontal electrophoresis separation, and wherein the system and device of the present invention providing unanticipated and nonobvious combination of features distinguished from the devices, apparatuses, inventions and methods preexisting in the art. The applicants are unaware of any device, apparatus, method, disclosure or reference that discloses the features of the claims and embodiments disclosed herein, and as more fully described below.

### IV. SUMMARY OF THE INVENTION

In one embodiment, a high-throughput multiplexing electrophoretic gel apparatus is disclosed. The high-throughput multiplexing electrophoretic gel apparatus is adapted and configured to provide a robust, auto or robotic-loadable, precast gel matrix, and is commercially-available and compatible for operational use with standard western blotting systems, and facilitates robust electrophoresis. The apparatus of the present invention comprises a gel casting device comprising a top portion and a bottom portion or base.

According to one embodiment, the top portion and base are separate pieces adapted to be aligned obversely and detachably connected in a closed position. The top portion and base are adapted and configured to be detachably connected in an intimate and complementary, co-planar relationship forming an interior, gel casting chamber.

The gel casting device further comprises at least one loading port through which an unpolymerized, flowable gel solution is introduced into the casting chamber. The upper surface of the base includes a plurality of protrusions which interpolate the gel solution as the gel solution is loaded into the casting chamber. After polymerization of the gel solution, a plurality of sample loading wells is formed integrally within the polymerized gel via the plurality of protrusions.

The vertical gel casting method is utilized to allow the gel solution to polymerize, thus the gel casting device is positioned in a vertical orientation for a standard polymerization period of time, and thereafter producing a polymerized gel layer comprising the plurality of integrally formed sample loading wells.

The plurality of sample loading wells is uniformly spaced and aligned in a geometrical arrangement of linear columns and horizontal rows, thereby enabling samples to be loaded into the plurality of wells via an automated microliter multi-pipette sample loading mechanism, such as the Opentrons® OT-2.

Thereafter, the sample-loaded polymerized gel layer is positioned atop a support base, and the support base and gel layer are placed horizontally in a horizontal electrophoresis tank in order to undergo horizontal electrophoresis separation of molecules. The receptacle of the electrophoresis tank is filled with a buffer solution to a level sufficient to fully immerse the sample-loaded polymerized gel layer. Next, an electric field is applied to the buffer solution so that an electric current passes through the buffer solution, and the sample-loaded polymerized gel layer for a period of approximately thirty to forty-five minutes.

### V. BRIEF DESCRIPTION OF THE DRAWING(S)

The advantages and features of the present invention will become better understood with reference to the following more detailed description and claims taken in conjunction with the accompanying drawings, in which like elements are identified with like symbols, and in which:

FIG. 1 is a front perspective view of a high-throughput multiplexing electrophoretic gel apparatus, in accordance to one embodiment of the present invention;

FIG. 2 is a top plan view of a top portion and base of a gel casting device, in accordance to one embodiment of the present invention;

FIG. 3 is a top plan view of a top portion and base of a gel casting device, in accordance to another embodiment of the present invention;

FIG. 4 is an end view of the gel casting device illustrating two loading ports, in accordance to one embodiment of the present invention;

FIG. 4a is an exploded perspective view of a gel casting device for conducting horizontal gel casting, in accordance to another embodiment of the present invention;

FIG. 4b is a bottom perspective view of the top portion of the gel casting device of FIG. 4a;

FIG. 5 is a top plan view of a gel casting device shown in an opened condition, in accordance to an alternate embodiment of the present invention;

FIG. 6 is an enlarged, partial cross-sectional view of the base showing the plurality of protrusions thereof interpolated through gel solution, in accordance to one embodiment thereof;

FIG. 6a is an enlarged, partial cross-sectional view of a separation medium depicting sample loading wells formed integrally therein, in accordance to one embodiment of the present invention;

FIG. 7 is a top perspective view of a support base supporting a polymerized gel layer disposed with a plurality of sample loading wells, in accordance to one embodiment of the present invention;

FIG. 8 is a perspective view of a conventional laboratory spatula;

FIG. 9 is a front, perspective view of an automated microliter multi-pipette sample loading mechanism;

FIG. 10 is a top perspective view of a horizontal electrophoresis tank, in accordance to one embodiment of the present invention;

FIG. 11 is a top perspective view of a digital scanning and imaging device;

FIG. 12 is a digital image depicting a resultant set of molecular weight ladders, in accordance to one embodiment of the present invention;

FIG. 13 is a digital image showing the resultant separation of U87 cell lysates and intermittent ladder, in accordance to one embodiment of the present invention;

FIG. 14 is a digital image illustrating resultant bands, some of which highlighted with molecular mass measures adjacent thereto, following the transfer of size-separated proteins onto a membrane incubated for beta-actin, and thereafter scanned and imaged, in accordance to one embodiment of the present invention;

FIG. 15 is a digital image showing the metrics utilized for assessing electrophoresis performance, in accordance to one embodiment of the present invention;

FIG. 16 is a digital image illustrating a resultant semi-regular molecular weight ladder following the loading of protein ladder samples into a plurality of sample loading wells in a separation medium, and thereafter being subjected to electrophoresis separation, in accordance to one embodiment of the present invention;

FIG. 17 is an inset detailed view of one set of the separated samples of FIG. 16, wherein the molecular mass measure is provided adjacent each highlighted band, in accordance to one embodiment of the present invention;

FIG. 18 illustrates a commercially-available kit, in accordance to another embodiment of the present invention; and

FIG. 19 depicts an alternate embodiment of the present invention.

### VI. DETAILED DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, the usage of the phrases “example embodiments”, “some language, throughout this embodiments”, or other similar that a particular feature, specification refers to the fact structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

For purposes of this disclosure, the term “multiplex” or “multiplexing” is intended to be defined as allowing multiple targets quantification in a single sample. For example, multiplex protein assays allow multiple targets quantification in a single sample.

In addition, for purposes of this disclosure, the term “high-throughput”, is defined as an analysis of hundreds or thousands of samples per day in a given laboratory or on a particular instrument.

Further, for purposes of this disclosure, the terms “robust” or “robustness” means the measure of an analytical procedure's capacity to remain unaffected by small, but deliberate variations in method parameters and provides an indication of its reliability during normal usage.

Still further, for purposes of this disclosure, the term “system” means a combination of one or more groups of elements configured to perform one or more functions.

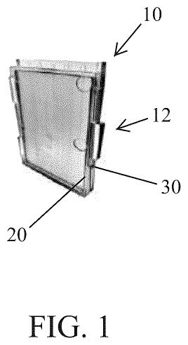

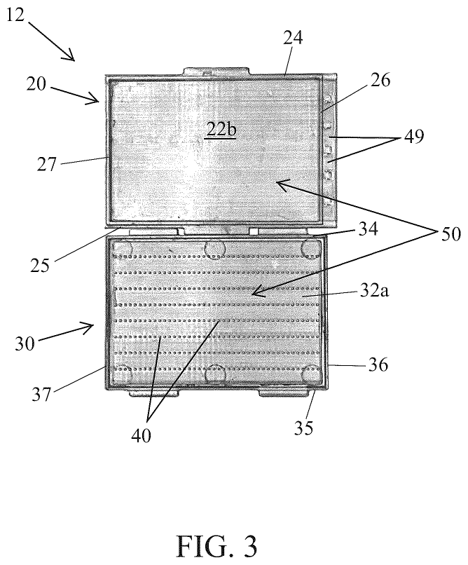

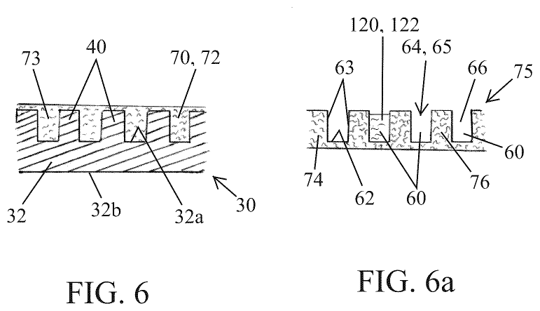

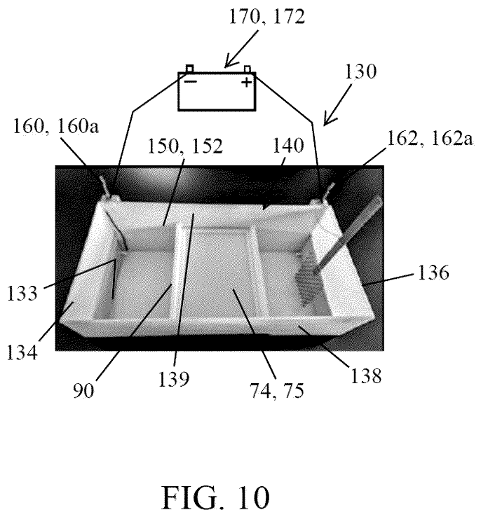

Consistent with the illustrations appended hereto, as embodied in FIGS. 1-7, 10, and 18-19, a high-throughput multiplexing electrophoretic gel apparatus, generally designated at 10 is disclosed, in accordance to one embodiment of the present invention. The high-throughput multiplexing electrophoretic gel apparatus, hereinafter “apparatus 10”, is adapted and configured to provide a robust, manual or robotic-loadable, precast gel matrix, and is commercially-available and compatible for operational use with standard western blotting systems, and facilitates robust electrophoresis.

Referring now more particularly to FIGS. 1-4, 6, and 6a, in accordance to one embodiment, the apparatus 10 comprises a gel casting device 12 comprising a generally square or rectangular configuration constructed of a lightweight, rigid material, such as a plastic polymer.

The gel casting device 12 imparts unanticipated and nonobvious functionality to the present invention. More particularly, the gel casting device 12 is adapted and configured to produce an optimum separation medium (gel matrix) for conducting horizontal electrophoresis separation of molecules, particularly sodium dodecyl-sulfate denatured (SDS-denatured) proteins. The separation medium may be integrated with ≥300 sample loading wells (to be described later in greater detail), wherein each loading well is adapted and configured to be loaded with a significantly lower volume of a liquid sample than previously utilized or developed in the art.

In addition, the sample loading wells are uniformly spaced and aligned in a geometrical arrangement of linear columns and horizontal rows within the separation medium, thereby enabling all loading wells to be loaded with samples simultaneously via an automated microliter multi-pipette sample loading mechanism.

Further, separation of the molecules of the loaded samples in the wells of the separation medium may be executed by fully immersing the separation medium in a horizontal electrophoresis tank filled with a buffer solution, and thereafter conducting horizontal electrophoresis.

The gel casting device 12 comprises a top portion 20 and a bottom portion or base 30. The top portion 20 comprises a planar top wall 22 having an upper surface 22a opposing a lower surface 22b from which a first longitudinal sidewall 24 opposing a second longitudinal sidewall 25, and a first latitudinal sidewall 26 opposing a second latitudinal sidewall 27 extend integrally downward about a perimeter of the lower surface 22b forming a continuous wall 28. The top wall 22 integrally extends outwardly past first latitudinal sidewall 26 a short distance forming an eave 23, wherein the eave 23 functioning as a handle 23a by which the user may more easily manipulate the gel casting device 12 between open and closed positions.

The base 30 comprises a bottom wall 32 having a lower surface 32b opposing an upper surface 32a from which a first longitudinal sidewall 34 opposing a second longitudinal sidewall 35, and a first latitudinal sidewall 36 opposing a second latitudinal sidewall 37 extend integrally upward about a perimeter of upper surface 32a forming a continuous wall 38.

According to one embodiment, the top portion 20 and base 30 are separate pieces adapted to be aligned obversely and detachably connected in a closed position. The top portion 20 and base 30 are adapted and configured to be detachably connected in an intimate and complementary, co-planar relationship forming an interior, separation medium casting chamber 50. The exterior surface 28a of the continuous wall 28 of the top portion 20 slidably engages the interior surface 38a of the continuous wall 38 of the base 30 in a snug, complementary-fit manner, whereby the top portion 20 is detachably connected to the base 30 via a firm frictional interference fit. In the closed position, the frictionally-engaged continuous walls 28 and 38 of the top portion 20 and base 30, respectively, form a substantially sealed continuous lip edge 54. For purposes of this disclosure, the terms “snug”, “snug-fit”, and “snugly” are each defined as a substantially-intimate, close-fitting relationship.

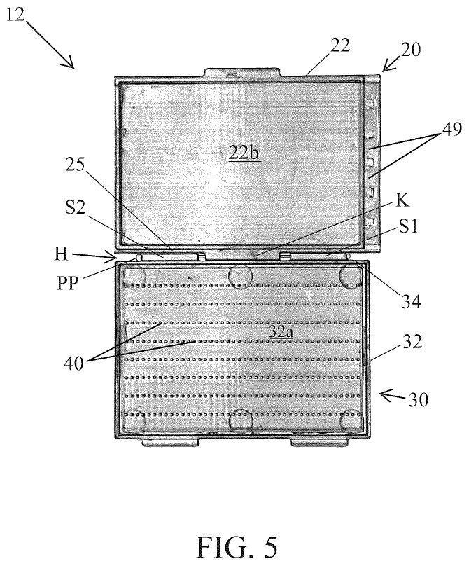

In accordance to another embodiment depicted in FIG. 5,

the top portion 20 and base 30 may be hingedly coupled via a hinge mechanism H, wherein the hinge mechanism H may comprise a knuckle K, a pair of shoulders S1 and S2, and a pivot pin PP. The shoulders S1 and S2 project integrally outward, coplanar to the first longitudinal sidewall 34 of the base 30, the shoulders S1 and S2 being aligned spatially parallel. The knuckle K projects integrally outward, coplanar to the second longitudinal sidewall 25 of the top portion 20, and is hingedly coupled between the pair of shoulders S1 and S2. The knuckle K of top portion 20 is hingedly coupled to the pair of shoulders S1 and S2 of the base 30 via the pivot pin PP. The pivot pin PP extends axially through a central hole in the first shoulder S1, through a central hole in the knuckle K, and through a central hole in the second shoulder S2, thereby hingedly coupling top portion 20 to the base 30.

The gel casting device 12 further comprises at least one loading port 49 through which an unpolymerized, flowable separation medium 70 (gel solution) is introduced into the casting chamber 50 of the gel casting device 12 within which the gel solution 70 (i.e., a polyacrylamide gel 72) undergoes vertical gel casting to form a polymerized gel layer 74 in the casting chamber 50. In FIG. 1, the gel casting device 12 is shown positioned in a vertical gel casting orientation.

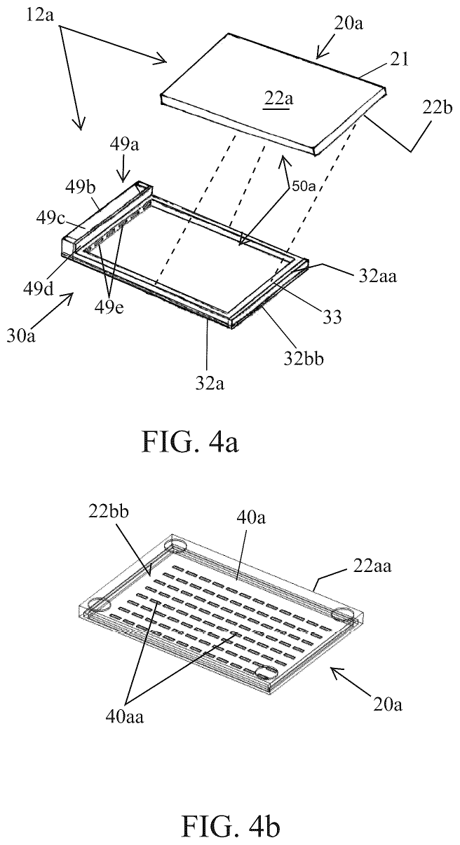

In reference to FIGS. 4a and 4b, and in accordance to another embodiment, a gel casting device 12a for conducting horizontal gel casting is depicted. The gel casting device 12a comprises a generally square or rectangular configuration constructed of a lightweight, rigid material, such as a plastic polymer. The gel casting device 12a comprises a top portion 20a and a bottom portion or base 30a. The base 30a comprises a bottom wall 32a having an upper surface 32aa opposing a lower surface 32bb. The upper surface 32aa of the bottom wall 32a of base 30a comprises a continuous upright wall 33, inset from a perimeter of the upper surface 32aa of the bottom wall 32a. The continuous upright wall 33 encloses a recessed cavity 33a providing a gel molding tray.

The top portion 20a and base 30a are adapted and configured to be detachably connected in an intimate and complementary, co-planar relationship forming an interior, separation medium casting chamber 50a, and which positions the gel casting device 12a in a closed condition.

One end of the bottom portion 30a comprises at least one loading port 49a through which an unpolymerized, flowable separation medium 70 (gel solution) is introduced into the casting chamber 50a of the gel casting device 12 within which the gel solution 70 (i.e., a polyacrylamide gel 72) undergoes vertical gel casting to form a polymerized gel layer 74. In accordance to one embodiment, the at least one loading port 49a comprises an elongated trough 49b having an open top 49c and a sidewall 49d, the sidewall 49d continguous to continuous upright wall 33. The sidewall 49d of trough 49b comprises a series of outlets 49e through which an unpolymerized, flowable separation medium 70 (gel solution) flows into the casting chamber 50a. The trough 49b and series of outlets 49e are in direct, open fluid communication with the casting chamber 50a.

The top portion 20a comprises a top wall 21 comprising an upper surface 22aa opposing a lower surface 22bb. The lower surface 22bb comprises a recessed platform 40a downwardly protruding therefrom. A plurality of protrusions 40aa projects integrally downward from the lower surface 22bb of the platform 40a.

Positioning of top portion 20a and base 30a in a closed condition such that the lower surface 22bb outer periphery edge of the top portion 20a is firmly engaged against the outer periphery edge of the upper surface 32aa of base 30a forms an air-tight sealed lip edge.

Upon loading of the unpolymerized, flowable gel solution 70 through the at least one loading port 49a and into the casting chamber 50a, the plurality of protrusions 40aa interpolate the unpolymerized, flowable gel solution 70. The flowable gel solution 70 comprises a polyacrylamide gel 72, wherein the polyacrylamide gel 72 comprises 10% polyacrylamide tris-glycine gel solution 73, or similar gel solution. Following a standardized polymerization time of sixty minutes, polymerization of the gel solution 70 is effectuated. Significantly, after polymerization of the gel solution 70, a plurality of sample loading wells 60 is formed integrally within the polymerized gel via the plurality of protrusions 40aa. Polymerization of the gel solution 70 produces a polymerized gel layer 74, the polymerized gel layer 74 provides a separation medium 75 for conducting horizontal electrophoresis separation of SDS-denatured proteins.

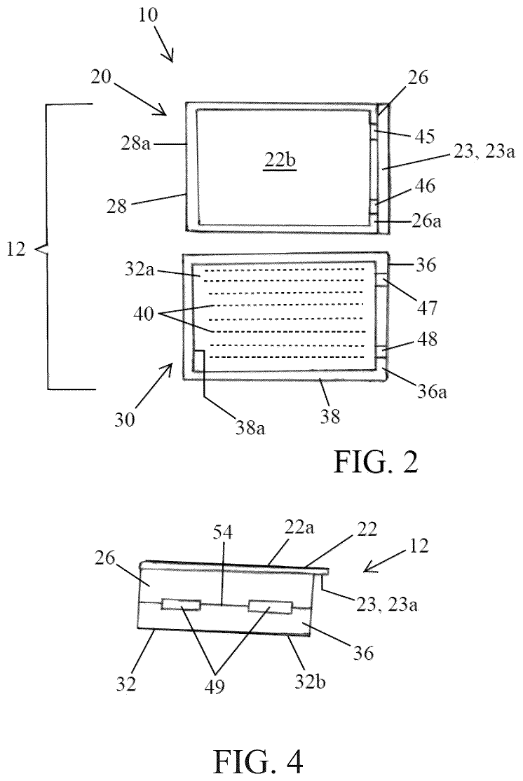

In accordance to one embodiment depicted in FIGS. 2 and 4, a pair of spaced recesses 45 and 46 is disposed along the bottom edge 26a of the first latitudinal sidewall 26 of the top portion 20, and a pair of complementary spaced recesses 47 and 48 is disposed along the upper edge 36a of the first latitudinal sidewall 36 of the base 30. Spaced recesses 45 and 46 and complementary spaced recesses 47 and 48 conjunctively provide a plurality of loading ports 49. More specifically, when the top portion 20 and base 30 are arranged in the closed position (as shown in FIG. 4), spaced recesses 46 and 45 are axially aligned with complementary spaced recesses 47 and 48, respectively, forming a plurality of loading ports 49. The plurality of loading ports 49 are in direct, open fluid communication with the casting chamber 50.

Referring now more particularly to FIGS. 2-3, and 5-6a, a plurality of protrusions 40 integrally projects upwardly from the upper surface 32a of the base 30. Upon loading of the unpolymerized, flowable gel solution 70 through the at least one loading port 49 and into the casting chamber 50, the plurality of protrusions 40 interpolate the unpolymerized, flowable gel solution 70. solution 70 comprises The flowable gel a polyacrylamide gel 72, wherein the polyacrylamide gel 72 comprises 10% polyacrylamide tris-glycine gel solution 73, or similar gel solution. Following a standardized polymerization time of sixty minutes, polymerization of the gel solution 70 is effectuated. Significantly, after polymerization of the gel solution 70, a plurality of sample loading wells 60 is formed integrally within the polymerized gel via the plurality of protrusions 40.

Polymerization of the gel solution 70 produces a polymerized gel layer 74, the polymerized gel layer 74 provides a separation medium 75 for conducting electrophoresis separation of SDS-denatured proteins. The polymerized gel layer 74, as previously described, comprises a polyacrylamide gel 72, wherein the polyacrylamide gel 72 comprises 10% polyacrylamide tris-glycine gel matrix 76. The 10% polyacrylamide tris-glycine gel matrix 76 comprises a plurality of sample loading wells 60 formed therein. The polymerized gel layer 74 (gel matrix 76) comprises a thickness measuring in a range comprising approximately 0.50 millimeters (mm) to 2.00 mm, in a range preferably comprising approximately 0.70 mm to 0.90 mm, and most preferably comprising 0.80 mm.

The plurality of sample loading wells 60 is uniformly spaced and aligned in a geometrical arrangement of linear columns and horizontal rows. In accordance to another embodiment, the plurality of sample loading wells 60 may be non-uniformly spaced and aligned. Each of the plurality of sample loading wells 60 comprises a closed bottom 62 opposing an open top 64. A continuous, inner circumferential sidewall 63 extends upward integrally from a periphery of the closed bottom 62 and terminates at the open top 64 forming a generally cylindrical space 66 for receiving a volume of a sample 120. The open top 64 provides an inlet aperture 65 through which a sample 120 is loaded. Each well 60 comprises a sample loading volume measuring in a range comprising approximately 0.50 microliters (μl) to 10.00 μl, in a range preferably comprising approximately 0.75 μl to 5.25 μl, and most preferably comprising approximately 1.00 μl.

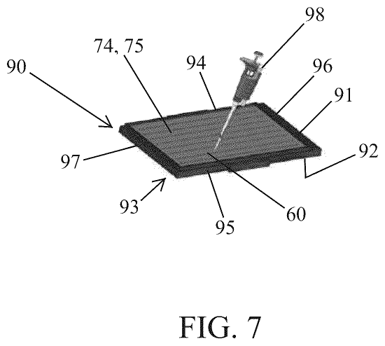

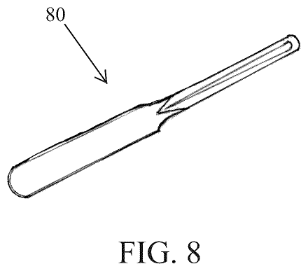

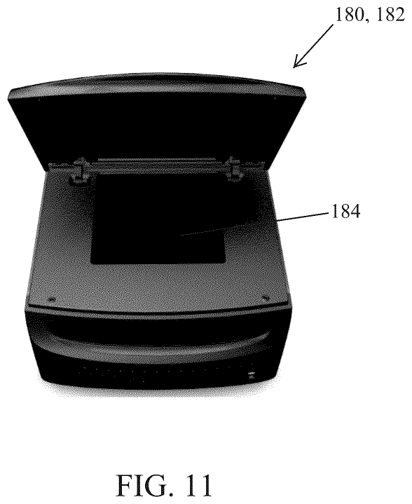

Referring now to FIGS. 7-11, the polymerized gel layer 74 comprises a number of sample loading wells 60, wherein the number of sample loading wells 60 comprises a number in a range comprising approximately 12 to 600, and in a range preferably comprising approximately 48 to 384. Most preferably, the polymerized gel layer 74 comprises at least 96 sample loading wells 60. In reference to FIG. 7, a polymerized gel layer 74 comprising 336 sample loading wells 60 is depicted, wherein each sample loading well 60 therein comprises a sample loading volume measuring approximately 1.00 μl.

The polymerized gel layer 74 is removed from the base 30 using a conventional laboratory spatula 80 and placed superjacent a support base 90, such that the inlet apertures 65 of the sample loading wells 60 of the gel layer 74 are facing upward. The support base 90 comprises a generally square or rectangular configuration constructed of a lightweight, rigid material, such as a plastic polymer. The support base 90 further comprises a planar top wall surface 91 opposing a planar bottom wall surface 92, the planar top wall surface 91 and planar bottom wall surface 92 joined integrally by a plurality of sidewalls 93, wherein the plurality of sidewalls 93 comprises a first longitudinal sidewall 94 opposing a second longitudinal sidewall 95, and a first latitudinal sidewall 96 opposing a second latitudinal sidewall 97. The planar top wall surface 91 defines a surface area dimensionally sized to accommodate the polymerized gel layer 74 within the confines of the periphery of the planar top wall surface 91.

Samples may be manually loaded into the sample loading wells 60 individually using a manual micropipette instrument 98, as shown in FIG. 7. However, hand loading hundreds of sample loading wells 60 manually is tedious, time-intensive, and error-prone.

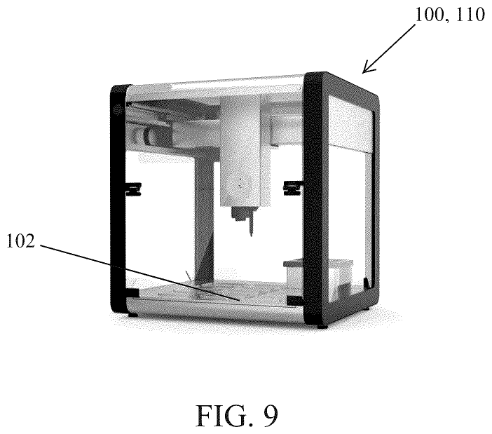

Preferably, samples 120 are loaded via an automated microliter multi-pipette sample loading mechanism 100, such as the Opentrons® OT-2 110 illustrated in FIG. 9. The support base 90 is dimensionally sized, shaped, and configured to sit atop the deck 102 of the automated microliter multi-pipette sample loading mechanism 100 in a size-fit or size-accommodating manner. The uniformly spaced and geometrically aligned arrangement featured by the sample loading wells 60 conforms with spaced intervals between tips on the multi-pipette sample loading mechanism 100, where such loading mechanisms 100 employ a multi-channel pipette, thereby enabling simultaneous loading of samples 120 in all sample loading wells 60 disposed in the separation medium 75 (10% polyacrylamide tris-glycine gel matrix 76) in a quick, easy, consistent, error-free, and efficient manner.

In accordance to one exemplary embodiment, after seating the support base 90 superjacent the deck 102 of the automated microliter multi-pipette sample loading mechanism 100, liquid samples 120 are loaded into each of the plurality of sample loading wells 60 via the loading mechanism 100. Each liquid sample 120 comprises a volume of 1.00 μl of SDS-denatured protein sample 122. Thus, 1.00 μl of SDS-denatured protein sample 122 is loaded into each sample loading well 60 via the loading mechanism 100.

In order to more easily visualize and quantify the protein bands, a fluorescent protein ladder mixture of proteins pre-stained with fluorescent dye is loaded into each of the sample loading wells 60. The bands fluoresce when the light of a specific wavelength falls on them. Molecular weight markers, or ladders, are a set of standards that are used for determining the approximate size of a protein or a nucleic acid fragment run on an electrophoresis gel. Protein ladders are used to help estimate the size of proteins separated during electrophoresis. They serve as points of reference because they contain mixtures of highly purified proteins with known molecular weights and characteristics. Molecular weight markers or protein ladders also aid in orienting the gel or membrane quickly and monitoring gel migration.

In addition, protein samples prepared for SDS-polyacrylamide gel electrophoresis analysis are denatured by heating in the presence of a sample loading buffer. One exemplary sample buffer is Tris-Glycine sample buffer. Tris-Glycine sample buffer comprises Tris HCl (63 millimoles (mM)), glycerol concentration (10%), SDS (2%), and bromophenol blue concentration (0.0025%), and pH measure of 6.8.

Another exemplary sample buffer is Tris Tricine sample

buffer. Tris Tricine sample buffer comprises Tris HCl (450 mM), glycerol concentration (12%), SDS (4%), Coomassie Blue G concentration (0.00075%), phenol red concentration (0.0025%), and pH measure of 8.45.

According to one embodiment, the 1.00 μl SDS-denatured protein sample 122 comprises 2.5 mg/ml of coomassie blue solution ladder. After all sample loading wells 60 have been loaded by the Opentrons® OT-2 110 robots, or manually loaded, the support base 90 with sample-loaded polymerized gel layer 74 positioned thereatop, is placed in a horizontal electrophoresis tank 130. The horizontal electrophoresis tank 130 comprises a bottom wall 132 having a perimetric interface 133 from which a right sidewall 136, a left sidewall 134, a forward sidewall 138, and a rear sidewall 139 extend upward integrally enclosing the bottom wall 132 and forming a receptacle 140 configured to contain a buffer solution 150. The horizontal electrophoresis tank 130 is structurally configured, sizably-shaped, and dimensioned to receive the support base 90 with sample-loaded polymerized gel layer 74 therein in a size-fit manner.

The support base 90 with sample-loaded polymerized gel layer 74 is positioned horizontally atop the bottom wall 132 of the tank 130, in a plane transverse to the bottom wall 132 of the tank 130. The receptacle 140 is then filled with buffer solution 150 to a level sufficient to fully immerse the sample-loaded polymerized gel layer 74 therein. Thereafter, an electric field is applied to the buffer solution 150 so that electric current passes through the buffer solution 150, SDS-denatured protein samples 122, and polymerized gel layer 74.

In order to provide the electric field required to drive electrophoresis separation of molecules, two conductive electrodes 160 and 162, connected electrically to an external power source 170, are coupled to the tank 130 at opposing ends thereof. The two conductive electrodes 160 and 162 comprise a cathode platinum wire 160a and a platinum-coated titanium anode plate 162a. It is envisioned the two conductive electrodes 160 and 162 may also be constructed of other suitable corrosion and/or degradation resistant metals and alloys, such as stainless steel. The electrical power source 170 comprises a 100 volts (V) direct current (DC) power supply 172. The cathode platinum wire 160a and platinum-coated titanium anode plate 162a is immersed in the buffer solution 150. According to one embodiment, the buffer solution 150 comprises Tris (hydroxymethyl) aminomethane hydrochloride 152.

In order to effect the electrophoresis separation, two opposite ends of the sample-loaded polymerized gel layer 74 (separation medium 75) are exposed to the buffered solution 150. Thereafter, the electrophoretic molecular separation process is triggered by activating the 100V DC power supply 172, thereby applying an electric field to the sample-loaded polymerized gel layer 74. The electrophoretic separation process is conducted approximately 30 to 45 minutes.

After completion of the electrophoretic separation test, the sample-loaded polymerized gel layer 74 is removed from the support base 90 via a conventional laboratory spatula 80 and placed on the interface 184 of a digital scanning and imaging device 180 to detect, identify, and visually produce infrared fluorescence of the molecular weight ladder bands as digital images. In accordance to one embodiment, the digital scanning and imaging device 180 is a LI-COR® Odyssey® Imaging System 182.

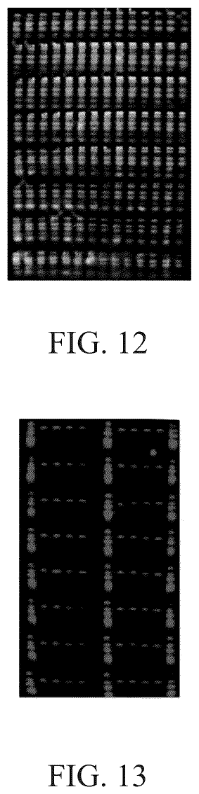

In FIG. 12, the results of the separation of the bands corresponding to the 1.00 μl coomassie blue solution (2.5 mg/ml) ladder samples can be observed. The resultant set of molecular weight ladders illustrated therein was identified following immersed horizontal electrophoresis separation.

In accordance to another embodiment, 1.00 μl of bromophenol blue protein ladder is loaded via the Opentrons® OT-2 device 110 into each of the plurality of sample loading wells 60 of the separation medium 75 (10% polyacrylamide tris-glycine gel matrix 76). The gel matrix 76 is placed in a horizontal electrophoresis tank 130 containing buffer solution 150, wherein the gel matrix 76 being fully immersed in the buffer solution 150. The buffer solution 150 comprises Tris (hydroxymethyl) aminomethane hydrochloride 152. Electrophoresis is triggered by activating the 100V DC power supply 172, thereby applying an electric field to the sample-loaded gel matrix 76. The electrophoretic separation process is conducted approximately 30 to 45 minutes.

In order to normalize the levels of protein detected, a loading control may be utilized and introduced with the samples 120. The loading control operates by confirming that both protein sample loading and protein transfer is equivalent across the polymerized gel matrix 76. The loading control may be selected from the group which includes, but is not limited to, alpha-tubulin and beta-actin (beta-actin).

In accordance to another exemplary embodiment, a number of U87 whole cell lysates and intermittent ladder were loaded via an automated microliter multi-pipette sample loading mechanism 100 into 96 sample loading wells 60 of a polymerized gel matrix 76. The U87 whole cell lysates are provided as a western blotting positive control. The cells are lysed using a Radio-Immunoprecipitation Assay (RIPA) lysis buffer. This process was repeated 16 times, and each sample loaded polymerized gel matrix 76 was subjected to fully immersed horizontal electrophoresis via the horizontal electrophoresis tank 130. Electrophoresis separation was conducted approximately 30 to 45 minutes. Next, the size-separated proteins were transferred onto a membrane, wherein the membrane was blotted for a loading control, namely, (alpha-tubulin), and thereafter scanned and imaged via a digital scanning and imaging device 180, wherein the selected digital scanning and imaging device 180 being the LI-COR. Odyssey® Imaging System 182. Alternatively, this step may be described as incubating the membrane with an antibody (ies) recognizing proteins-of-interest and detection antibodies by identifying and isolating each sample loading well 60 by converging on a region of interest, and thereafter scanning and imaging the membrane via a digital scanning and imaging device 180. FIG. 13 is a digital image showing the resultant separation of the U87 cell lysates and intermittent ladder, wherein reproducibility across blocks may be observed.

It is recognized that in the event a running buffer is used to completely submerge the gel matrix 76 when conducting fully immersed horizontal electrophoresis separation, and since the samples 120, 122 are positioned on the top, the inlet aperture 65 of each facing upward, sample wash out may be encountered. To prevent or otherwise eliminate sample wash out, the sample buffer may be adjusted to contain, for example, a higher glycerol percentage to promote higher density and facilitate sinking by the samples 120, 122 into corresponding wells 60.

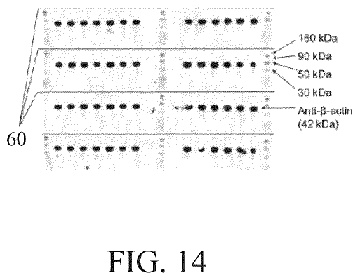

In accordance to another exemplary embodiment, ˜1.00 μl of a lysate was loaded via the automated microliter multi-pipette sample loading mechanism 100 into each of the plurality of sample loading wells 60 of a representative gel matrix 76. The gel matrix 76 was subjected to electrophoresis. The size-separated proteins were transferred onto a membrane, wherein the membrane was incubated for a loading control, namely, beta-actin, and thereafter scanned and imaged via a digital scanning and imaging device 180. FIG. 14 illustrates the resultant bands, some of which highlighted with molecular mass measures adjacent thereto.

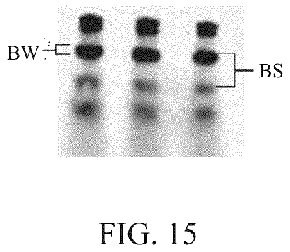

In reference to FIG. 15, the pattern of band separation is a core factor dictating the robustness of electrophoresis. Two key features utilized as metrics for analyzing band separation patterns are band widths and band separation. Band width BW is the measure of the vertical length of each molecular weight band in every lane. Band separation BS is a measure of the vertical length from the midpoint of one band to the midpoint of the next band. FIG. 15 is a digital image showing the band width BW and band separation BS of a molecular weight marker.

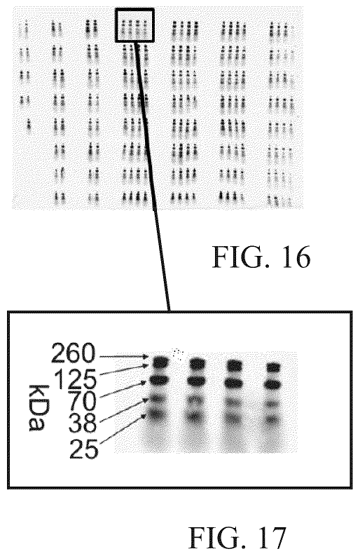

In reference to FIG. 16, a digital image illustrates a resultant semi-regular molecular weight ladder following the loading of protein ladder samples into a plurality of sample loading wells 60 in a separation medium, and thereafter subjected to electrophoresis separation.

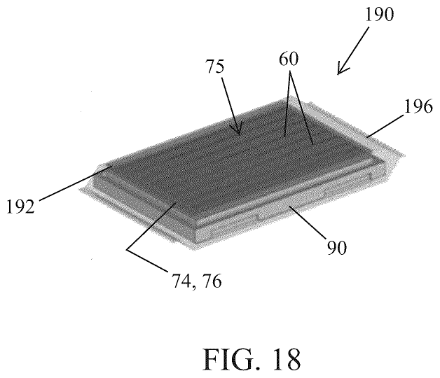

FIG. 17 is an inset detailed view of one set of the separated samples of FIG. 16. The molecular mass measure kiloDalton (kDa) (as opposed to molecular weight measure) is provided adjacent each highlighted band.

Referring now to FIG. 18, it is envisioned a support base 90, a customized polymerized gel layer 74, and a removably securable cover 192 may be made commercially-available, sold, and shipped as a kit 190. The customized polymerized gel layer 74 comprises a gel being suitable or felicitous to provide a separation medium 75 for conducting electrophoresis separation of molecules, particularly SDS-denatured proteins. In accordance to one embodiment, a suitable gel adapted and configured to provide a separation medium 75 for conducting electrophoresis separation of molecules includes, but is not limited to, 10% polyacrylamide tris-glycine gel 76. The gel 76 includes a plurality of sample loading wells 60, wherein a variety of numbers of wells 60 being available, and also customized in number, thereby being available in a number as selectively-desired and requested by a user/purchaser.

It is envisioned the gel 76 comprises a thickness measuring in a range comprising approximately 0.50 mm to 1.50 mm.

It is further envisioned each well 60 comprises a sample loading volume measuring in a range comprising approximately 0.50 μl to 10.00 μl.

It is still further envisioned the gel 76 comprises a number of sample loading wells 60, wherein the number of sample loading wells 60 comprises a number in a range comprising approximately 10 to 600, and in a range preferably comprising approximately 48 to 384. Most preferably, the polymerized gel 76 comprises approximately 96 sample loading wells 60.

The cover 192 is constructed of a rigid or semi-rigid plastic material and sized so as to effectively cover and shield completely the gel 76 prior to use. Cover 192 is removably attached to the support base 90 via a snap-fit arrangement, frictional-interferential fit, bolts and nuts, screws, clamps, hook-and-loop fasteners, or other suitable attachment mechanism or fastening means.

The support base 90, gel 76, and cover 192 are enclosed or otherwise enveloped entirely and sealed via plastic wrapping 194.

In accordance to still another embodiment of the present invention, a novel system and method for combining a protein-staining membrane with a polyacrylamide gel 72 (as previously described hereinabove regarding a number of exemplary embodiments) to simplify a laboratory technician's process for standard protein transfer from a polyacrylamide gel to a protein-staining membrane. The protein-staining membrane may be selected from the group which includes, but is not limited to, polyvinylidene fluoride (PVDF), Nitrocellulose, or other suitable material. The system and method comprises polyacrylamide gel, a protein-staining membrane, and a thin planar rigid membrane or substrate, such as a plastic card, wherein the card is characterized as a thin and rigid structure. The protein-staining membrane is placed atop the thin plastic card before unpolymerized polyacrylamide gel electrophoresis (SDS-PAGE) gel solution is poured into a gel casting device or apparatus. After being secured in the casting apparatus, the SDS-PAGE gel solution is poured into the casting device and allowed to polymerize to create the final concept.

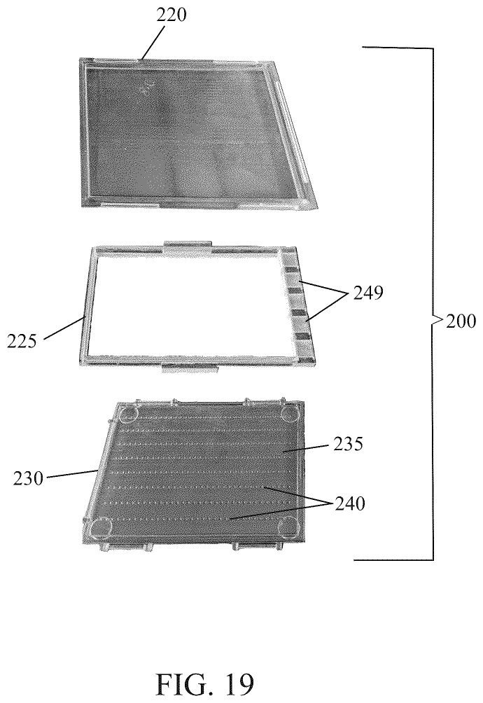

Finally, in reference to FIG. 19, an alternate embodiment of the present invention is depicted, wherein a gel casting device 200 comprises a three element structure comprising a top portion 220, a middle portion 225, and a base 230. The top portion 220, middle portion 225, and base 230 are connected in a stacked fashion so as to forms an interior, separation medium casting chamber. Placement of the device 200 in a stacked fashion also places the device 200 in a closed position, thereby forming an interior, separation medium casting chamber.

At least one loading port 249 is integrally formed along one of the latitudinal sidewalls of the middle portion 225. The at least one loading port 249 provides an opening through which an unpolymerized, flowable separation medium (gel solution) is introduced into the casting chamber of the gel casting device 200.

The base 230 comprises a sample wells molding portion 235 for forming a plurality of sample loading wells in the gel solution during polymerization thereof. The molding portion 235 comprises a plurality of protrusions 240 integrally projecting upwardly from the upper surface of the base 230. The top portion 220 and middle portion 225 enclose the gel solution, thereby providing a retaining mold within which polymerized gel is formed.

It is to be understood that the embodiments and claims are not limited in application to the details of construction and arrangement of the components set forth in the description and/or illustrated in drawings. Rather, the description and/or the drawings provide examples of the embodiments envisioned, but the claims are not limited to any particular embodiment or a preferred embodiment disclosed and/or identified in the specification. Any drawing figures that may be provided are for illustrative purposes only, and merely provide practical examples of the invention disclosed herein. Therefore, any drawing figures provided should not be viewed as restricting the scope of the claims to what is depicted.

The embodiments and claims disclosed herein are further capable of other embodiments and of being practiced and carried out in various ways, including various combinations and sub-combinations of the features described above but that may not have been explicitly disclosed in specific combinations and sub-combinations.

Accordingly, those skilled in the art will appreciate that the conception upon which the embodiments and claims are based may be readily utilized as a basis for the design of other structures, methods, and systems. In addition, it is to be understood that the phraseology and terminology employed herein are for the purposes of description and should not be regarded as limiting the claims.

Furthermore, the purpose of the foregoing Abstract is to enable the U.S. Patent and Trademark Office and the public generally, and especially including the practitioners in the art who are not familiar with patent and legal terms or phraseology, to determine quickly from a cursory inspection the nature and essence of the technical disclosure of the application. The

Abstract is neither intended to define the claims of the application, nor is it intended to be limiting to the scope of the claims in any way. It is intended that the application is defined by the claims appended hereto.

What is claimed is:

1. A method for producing a separation medium for electrophoresis separation, the method comprising the steps of:
connecting a top portion and a base of a gel casting device so as to place the gel casting device in a closed position, thereby forming at least one loading port;
loading a volume of gel solution through the at least one loading port and into an interior casting chamber of the gel casting device;
positioning the gel casting device in at least one of a horizontal orientation and a vertical orientation;
effectuating polymerization of the gel solution by maintaining the gel casting device in the vertical orientation for a period of approximately sixty minutes, thus forming a polymerized gel layer;
disconnecting the top portion from the base of the gel casting device so as to place the gel casting device in an open position;
removing the polymerized gel layer from the base with a laboratory spatula; and
placing the polymerized gel layer superjacent a support base.

2. A method for conducting horizontal electrophoresis separation of molecules, the method comprising the steps of:
removing a polymerized gel layer from a base of a gel casting device via a laboratory spatula;
placing the polymerized gel layer superjacent a support base, such that inlet apertures of sample loading wells of the polymerized gel layer face upward;
seating the support base superjacent a laboratory benchtop or a deck of an automated microliter multi-pipette sample loading mechanism;
inputting desired sample loading parameters into a manual micropipette or the automated microliter multi-pipette sample loading mechanism;
loading SDS-denatured protein samples into the sample loading wells via the manual micropipette or activating the automated microliter multi-pipette sample loading mechanism, thereby enabling simultaneous loading of SDS-denatured protein samples into the sample loading wells, thus providing a sample-loaded polymerized gel layer;
positioning the support base with sample-loaded polymerized gel layer horizontally atop a bottom wall of a horizontal electrophoresis tank;
filling a receptacle of the horizontal electrophoresis tank with a buffer solution to a level sufficient to fully immerse the sample-loaded polymerized gel layer; and
applying an electric field to the buffer solution so that an electric current passes through the buffer solution, and the sample-loaded polymerized gel layer for a period of approximately thirty to forty-five minutes.

3. The method of claim 2, further comprising the steps of:
removing the sample-loaded polymerized gel layer from the support base via the laboratory spatula; and
placing the sample-loaded polymerized gel layer atop an interface of a digital scanning and imaging device.

4. The method of claim 3, further comprising the steps of:
scanning the sample-loaded polymerized gel layer to detect and identify infrared fluorescence of molecular weight ladder bands; and
producing digital images of the infrared fluorescence of separated molecular weight ladder bands.

5. The method of claim 2, wherein the polymerized gel layer comprises polyacrylamide gel.
